(19) United States
(12) Patent Application Publication
(10) Pub. No.: US 2025/0128062 A1
(43) Pub. Date: Apr. 24, 2025

(54) System and Method for Reversible Sensory Nerve Block

(71) Applicant: University of Connecticut, Farmington, CT (US)

(72) Inventors: Bin Feng, Storrs, CT (US); Longtu Chen, Willington, CT (US); Jia Liu, Vernon, CT (US); Shaopeng Zhang, Manchester, CT (US)

(73) Assignee: University of Connecticut, Farmington, CT (US)

(21) Appl. No.: 18/924,049

(22) Filed: Oct. 23, 2024

Related U.S. Application Data
(60) Provisional application No. 63/592,343, filed on Oct. 23, 2023.

(51) Int. Cl. A61N 1/36; A61M 5/142; A61N 1/32; A61N 1/372

(52) U.S. Cl. CPC A61N 1/36057; A61M 5/14276; A61N 1/323; A61N 1/36071; A61N 1/36171; A61N 1/37211; A61M 2205/054

(57) ABSTRACT

System for effecting a reversible sensory block includes a hybrid neuromodulator and hybrid lead for delivering electrical and pharmacological stimulation. An implantable modulator provides electrical pulse generation and pharmacological fluid infusion. Method for effecting a reversible sensory block delivers low-frequency stimulation based on the conduction velocity of a target afferent. Afferents with different conduction velocities may be blocked by stimulation with different frequencies. Temporally synchronized pulse stimuli from multiple stimulating electrodes may interfere with each other to stimulate a specific afferent. Spatial summation of synchronized low-frequency pulse stimulation may be delivered at multi-sites. Temporal interference of two kilohertz sinusoidal stimuli with slight frequency difference deliver a low-frequency sinusoidal stimulus at a focal region of interference. Spatial summation translates to evoked action potentials that converge at T-junction of a targeted afferent. Pharmacological delivery of Na,K-ATPase inhibitors or antagonists to the DRG facilitates electrical stimulation for selective blockage of small-diameter nociceptor afferents.

### CROSS-REFERENCE TO RELATED APPLICATION

The present application is a non-provisional of and claims the benefit of U.S. Provisional Application No. 63/592,343, filed on Oct. 23, 2023. The entire content of the provisional patent application is incorporated herein by reference.

### STATEMENT REGARDING FEDERALLY SPONSORED RESEARCH

This invention was made with government support under DK120824 and NS113873 awarded by the National Institutes of Health, and 1844762 awarded the National Science Foundation. The government has certain rights in the invention.

### BACKGROUND OF THE INVENTION

### 2. Field of the Invention

Systems and methods are provided for reversible sensory nerve block by multi-modal peripheral neuromodulation, including electrical stimulation strategies of different paradigms and local chemical modulators to treat neurological disorders, including specifically chronic pain. In addition, reversible sensory nerve block is also applicable for treating, inter alia, neurogenic chronic cough, obesity, and fecal and urinary incontinence.

### 3. Description of Related Art

One in three adults in the U.S. suffers from at least one type of chronic pain condition, resulting in over $600 billion in expenditure for chronic pain treatment annually. This recurring high cost reflects the fact that existing pain treatments only temporarily manage symptoms but do not ‘cure’ the disease of chronic pain.

Acute pain is generally managed in a clinic setting by chemical nerve block techniques, e.g., via injection of local anesthetics. Chemical injection techniques are non-selective, and generally block both nociceptive (pain-related) and non-nociceptive sensory (afferent) neurons, as well as motor (efferent) axons in the nerve trunk. Chemical injection techniques do not offer a long-term solution for chronic pain conditions.

Mounting preclinical evidence reveals that central sensitization in the central nervous system (CNS) is mainly driven by enhanced spatial and temporal summation of synaptic input from peripheral C-fiber afferents, especially C-fiber nociceptors in deep tissues (see (Latremoliere and Woolf, 2009) for a review). Nociceptors include both myelinated fast-conducting Aδ-fiber afferents and unmyelinated slow-conducting C-fiber afferents, giving rise to sharp first pain and delayed second pain, respectively (Purves et al., 2001). Unlike the immediate sharp sensation from the first pain, the second pain is an achy, dull, throbbing, or burning sensation that often lingers beyond the brief stimulus that evokes it (Staud et al., 2007). Consistently, chronic pain patients often describe their pain as achy (53%), throbbing (28%), burning (22%), and/or dull (20%) (Jensen et al., 2013), indicating activation of C-fiber nociceptors. Peripheral sensitization leads to increased responses to noxious and non-noxious stimuli as well as the awakening of previously unresponsive, inactive ‘silent’ C-fiber nociceptors (Feng and Gebhart, 2011; Feng et al., 2012). Thus, nociceptor sensitization, especially C-fiber sensitization underlies prolonged pain sensation that outlasts stimulus duration and is likely a major mechanism driving the persistence of chronic pain. In addition, many chronic pain patients also report sharp sensations like stabbing, shooting, piercing, and zapping (Jensen et al., 2013; Dworkin et al., 2016), indicative of Aδ-nociceptor activation. Thus, effective treatment of chronic pain should aim to suppress or reverse the sensitization of both C- and Aδ-nociceptors, with an emphasis on C-fiber nociceptors. Also, the treatment needs to avoid affecting normal sensory, proprioceptive and motor functions, and thus should spare the large Aα- and Aβ-fiber afferents and efferents (motor fibers).

Current electrical stimulation protocols for blocking peripheral nerves implement high-frequency stimulation over 1000 Hz, e.g., kilohertz stimulation. Increased stimulus frequency entails shorter stimulus duration, and thus elevated threshold of stimulation according to the strength-duration relation. Thus, conventional high frequency stimulation techniques involve high power consumption and limit wider application to manage a range of symptoms.

EP 3932475 to Stylos et al. discloses systems and methods configured to rehabilitate or strengthen one or more muscles in a patient and to reduce pain sensed by the patient through electrical stimulation signals delivered to one or more target peripheral nerves. The Stylos '475 publication discloses delivering first stimulations signals having a first range of frequencies to target nerves to rehabilitate muscles innervated by the motor nerves, and second stimulations signals having a second range of frequencies to the target nerves to provide pain relief to the patient. The first range of frequencies is lower (2 Hz to 100 Hz) than the second range of frequencies (100 Hz to 10,000 Hz).

U.S. Pat. No. 9,248,289 to Bennett et al. systems and methods for treatment of pain associated with muscle and tendon by inducing action potentials in target neural structures that may be patterned to be biomimetic or stochastic. The stimulating can be a consistent and repeatable pattern or in a random fashion or a combination of repeatable and random patterns to generate a stochastic response of action potentials in the plurality of target afferent sensory fibers. The Bennett '289 patent discloses stimulation frequencies of 1 Hz-20 kHz.

However, the above stimulus strategies do not differentiate the targeting of large-myelinated A-fiber afferents from the small unmyelinated C-fiber afferents. As described in above, it is the C-fiber afferents that are the major drivers of the persistence of chronic pain. Nonselective electrical stimulation of the afferents at frequencies above 20 Hz will likely drive the activation of Aδ-nociceptors to be over the painful threshold leading to a perception of the “first pain”, a detrimental side effect that exacerbates the symptoms of chronic pain. In distinct contrast, the disclosed systems and methods combine electrical and pharmacological modulation of the peripheral afferent neurons, which not only selectively blocks the C-fiber nociceptors, but can also block Aδ-nociceptors at a low stimulation frequency (<15 Hz).

Also, conventional stimulus strategies do not offer spatial tunability of neuromodulation targets. Usually, it is the nervous tissues closer to the electrodes that get activated. In contrast, the disclosed systems and methods provide at least the following technical features to enable spatially selective and tunable activation of peripheral nerve axons and cell bodies: (i) synchronized multisite pulse stimulation, and (ii) temporal interference of two kilohertz sinusoidal stimulations with frequencies differing by 10 to 50 Hz (e.g., two sites operating at 2000 Hz and 2020 Hz, respectively).

Chronic pain is a disease in its own right and is featured with central sensitization in the spinal cord and brain, which results from long-lasting elevated afferent drives from the peripheral nervous system, especially from C-fibers innervating deep tissues. The disclosed systems and methods enable selective transmission block of small-diameter nociceptive afferents, especially C-fiber nociceptors to suppress their drive to the central nervous system, offering a direct ‘cure’ to the disease nature of chronic pain by reversing the central sensitization in the brain and the spinal cord.

### SUMMARY OF THE INVENTION

The disclosed systems and methods provide peripheral nerve stimulation that deliver reversible nerve block by sub-kilohertz electrical pulse stimulation from multiple sites (typically <5 Hz at each electrode lead). The disclosed systems and methods can also block the peripheral nerve by temporal interference of two kilohertz sinusoidal stimulations with frequencies differing by 10 to 50 Hz (e.g., 2000 and 2020 Hz, respectively). The systems and methods are effective in alleviating pain by blocking pain sensory transmission from the periphery.

In embodiments, a multichannel electrode array is provided that delivers spatially and temporally synchronized electrical stimulation, e.g., to the peripheral nerve trunk or the dorsal root ganglia (DRG) in the vertebral foramen to achieve selective afferent transmission block. The systems and methods are effective to provide peripheral nerve and DRG stimulations at multiple sites.

In embodiments, the nerve blocking effect of the disclosed sub-kilohertz electrical stimulation is reversible, e.g., neural transmission returns within five (5) minutes of discontinuance of the sub-kilohertz electrical stimulation.

In embodiments, a system for effecting a reversible sensory block includes a hybrid neuromodulator that includes a hybrid lead for delivering electrical stimulation and pharmacological stimulation. The system may further include an implantable modulator. The implantable modulator may be adapted for electrical waveform generation and pharmacological fluid infusion. The waveform includes, but is not limited to, pulses, sinusoids, trapezoids, saw-tooth, white noise, and other arbitrary waveforms.

In embodiments, a system for effecting a reversible sensory block includes a pump that delivers a fluid infusion. The pump may be a peristaltic pump or an osmotic pump.

In embodiments, a system for effecting a reversible sensory block includes a hybrid lead in the vertebral foramen associated with a nerve cuff.

In embodiments, a system for effecting a reversible sensory block includes a wireless remote controller adapted to communicate with a hybrid neuromodulator.

In embodiments, a method for effecting a reversible sensory block includes delivering low-frequency electrical pulse stimulation to block unmyelinated C-type afferents. The low-frequency stimulation may be at a frequency of less than 500 Hz, at a frequency of less than 10 Hz, or at a frequency of less than 5 Hz.

In embodiments, a method for effecting a reversible sensory block includes delivering multiple kilohertz sinusoidal stimulations with frequencies differing by 10 to 50 Hz. The temporal interference between any two of the multiple kilohertz stimulations causes a low-frequency ‘envelop’ sinusoidal stimulation of 10 to 50 Hz to effectively block unmyelinated C-fiber nociceptors at an adjustable focal region in the peripheral nerve and DRG.

In embodiments, a method for effecting a reversible sensory block delivers low-frequency stimulation that is selected based on the conduction velocity of a target afferent. In an embodiment, afferents with different conduction velocities are blocked by pulse or sinusoidal stimulation with different frequencies. In an embodiment, temporally synchronized stimuli from multiple stimulating electrodes interfere with each other to stimulate a specific afferent. In an embodiment, spatial summation of synchronized low-frequency stimulation is delivered at multi-sites along a nerve trunk. In an embodiment, the spatial summation translates to evoked action potentials that converge at a T-junction of a targeted afferent.

In embodiments, a method for effecting a reversible sensory block includes delivering low-frequency electrical stimulation and a pharmacological block to unmyelinated C-type and thinly myelinated Aδ-type sensory afferents. In embodiments, the pharmacological block is directed to the sodium, potassium ATPase at peripheral nerve trunk, the DRG, and/or dorsal roots. In embodiments, the pharmacological block is a Na, K-ATPase antagonist or inhibitor. The Na, K-ATPase antagonist may be digoxin, a cardiac glycoside (e.g., ouabain). In embodiments, the pharmacological block can be long-lasting by packaging the inhibitors of Na, K-ATPase into biocompatible drug-delivery vehicles, e.g., liposomes and poly(lactic-co-glycolic acid) (PLGA) microspheres.

In embodiments, the system and method may be used to generate a reversible sensory block for treatment of chronic pain. In embodiments, the chronic pain may be associated with chronic leg or arm pain, diabetic neuropathy, failed back surgery syndrome, complex regional pain syndrome, lower abdominal pain, and pelvic pain.

The disclosed systems and methods have wide ranging applicability for various pain symptoms, such as limb pain, neuropathic pain, small-fiber neuropathy.

In embodiments, the system and method may be used to block sensory nerves for treating chronic neurogenic cough, obesity, and fecal/urinary incontinence.

Additional advantageous features and functions of the disclosed systems and methods will be apparent from the detailed description which follows, particularly when read in conjunction with the appended figures.

### BRIEF DESCRIPTION OF THE FIGURES

To assist those of skill in the art in making and using the disclosed systems and methods, reference is made to the accompanying figures, wherein:

FIG. 1A is a plot of optimal blocking frequency (OBF) vs. conduction velocity (CV) for particular afferents.

FIG. 1B is a plot of transmission block probability vs. stimulating frequency for particular afferents.

FIG. 1C is a schematic depiction of a sensory afferent that include the peripheral and central axons, T-junction, stem axon, and the cell body in the DRG (soma). Electrical stimulation is delivered at multiple sites along the afferent, including the spinal nerve (SN), dorsal root (DR), DRG and T-junction. The protocol of electrical stimulation include 1) synchronized pulse stimulation (Syn-PS) from multiple sites and 2) temporal interference of sinusoidal stimulations (TI-SS).

FIG. 1D is a depiction of the Syn-PS stimulation protocol, which includes a spatial and temporal summation of low-frequency pulse stimulation at multiple sites of the peripheral nerve trunk. Syn-PS leads to equivalent stimulation with OBF at the T-junction of the sensory neurons in the dorsal root ganglia (DRG).

FIG. 1E is a depiction of the TI-SS stimulation protocol. The temporal summation of two kilohertz sinusoidal stimulations with a slight frequency difference (e.g., 2000 Hz and 2020 Hz) converges at focal regions, resulting in an amplitude-modulated kilohertz stimulation signal. Neurons filter out the kilohertz frequency and are activated by the low-frequency sinusoidal “envelope” stimulation (5 to 20 Hz). The focal region can be fine-tuned by adjusting the relative stimulus strength of the multiple sinusoidal stimulating signals.

FIG. 2A is a schematic depiction of a sensory afferent, including its peripheral endings that transduce stimuli into action potentials and its central projection into the spinal cord.

FIG. 2B is a plot of conduction delay vs. time during sub-kilohertz electrical pulse stimulation of the spinal nerve or DRG.

FIG. 3A is a series of plots showing a positive correlation between optimal blocking frequency (OBF) and conduction velocity (CV) for an afferent through single-fiber recordings from afferents with different CV, showing conduction block by different range of stimulus frequencies at the spinal nerve.

FIG. 3B is a plot showing that OBF increases with afferent CV.

FIG. 4A is a schematic depiction of a selective nerve trunk stimulation using a cuff electrode array and protocol with cuff electrodes placed on multiple locations along the spinal nerve.

FIG. 4B is a schematic depiction of the spatial distribution of areas affected by different combinations of electrical stimulation electrodes at the trunk of the peripheral nerve.

FIG. 4C are schematic depictions of an electrode array layout targeting the peripheral nerve trunk.

FIG. 5 is a schematic depiction of a hybrid reversible sensory block modality that includes an implantable hybrid neuromodulator that targets thoracolumbar and sacral DRG to treat chronic pain, and that delivers local electrical and pharmacological (inhibitor of the sodium, potassium ATPase, e.g. digoxin) modulation to epidural DRG, achieving reversible afferent transmission block in unmyelinated C-fiber and thinly myelinated Aδ-fiber nociceptors.

FIG. 6A is a schematic depiction of an ex vivo preparation for single-fiber recordings (“SN-stim” refers to spinal nerve stimulation).

FIG. 6B are plots of transmission block by DRG stimulation in an Aδ-fiber and a C-fiber (at 100 Hz and 50 Hz stimulation), with arrows indicating action potentials and arrow heads indicating an absence of AP transmission.

FIG. 6C is a plot showing progressive increase in conduction delay following DRG stimulation (“Rec” refers to recovery).

FIG. 7A is a schematic depiction of a set-up for in vivo single-fiber recordings from the L6 dorsal root in an isoflurane-anesthetized mice.

FIG. 7B is a further schematic depiction of the set-up shown in FIG. 7A.

FIG. 7C shows single-fiber recordings from the L6 dorsal root of a mouse using the experimental set-up of FIGS. 7A and 7B.

FIG. 8A is a schematic depiction of a neural membrane model that includes a soma, stem axon, T-junction, and peripheral and central axons.

FIG. 8B is a plot showing a more significant increase in intra-axonal cellular Na+ concentration ([Na⁺]ᵢ) and decrease in intra-axonal K+ concentration ([K⁺]ᵢ) at the onset of DRG stimulation in stem, central and peripheral axons than in soma (arrowheads). This dysregulated intra-axonal [Na₊]ᵢ and [K₊]ᵢ is restored by Na, K-ATPase activity 10 to 15 sec after the stimulation ends (arrows).

FIG. 8C is a plot showing regular conduction of action potentials before DRG stim (arrow) and conduction block within 10 sec after DRG stimulation (arrowhead).

FIG. 8D is a magnified view of the membrane potential at the time of action potential conduction and conduction block in FIG. 8C.

FIG. 9A is a plot of ex vivo single-fiber recordings from afferent axons in a 3-compartment tissue chamber that isolates the DRG from the spinal nerve and dorsal root allowing focused pharmacological application.

FIG. 9B are plots showing that transmission block is achieved in a C-fiber afferent by combined 10 Hz DRG stimulation with ouabain application (10 μg/mL or 17.1 μM).

FIG. 9C is a plot showing application of ouabain (17.1 μM) allows 10 Hz DRG stimulation to selectively block C-fiber afferents while leaving Aδ-fibers unaffected.

FIG. 10A is an immunobiological staining of Na,K-ATPase alpha1 subunit (NaKAα1) in mouse DRG sections (scale 200 μm).

FIG. 10B is an image showing that NaKAα1 expression is greater in large-diameter DRG neurons (cross-hatched arrows) as compared to small-diameter DRG neurons (non-cross-hatched arrows) (scale 50 μm). This result indicates that unmyelinated C-fiber afferents are more susceptible to activity-dependent transmission block that myelinated A-fiber afferents.

FIG. 10C is a magnified view in the box region shown in phantom in FIG. 10B (scale 2 μm).

FIG. 11A is a schematic of a hybrid electrical/pharmacological neuromodulator for targeted delivery of electrical stimulation and pharmacological antagonist to sodium, potassium ATPase to the DRG, spinal nerve and dorsal root. The system consists of an implantable modulator, hybrid lead and an external control unit. The implantable modulator communities with the external control unit wirelessly by near-field communication protocols for secure and fast two-way data transmission. The hybrid leads are placed in the vertebral foramen for delivery of electrical and chemical neuromodulation to the DRG, dorsal root and spinal nerve.

FIG. 11B is a depiction of fabrication of a hybrid lead that enables focal electrical and pharmaceutical neuromodulation. The electrode array is built by planar microfabrication to consist of multiple electrodes. The planar electrode array is then spiral wrapped around a fluid-delivery catheter to form the hybrid lead.

FIGS. 12A-12E are schematics of hybrid lead systems for delivering local electrical and pharmacological stimuli to the DRG, dorsal root and spinal nerve.

### DETAILED DESCRIPTION

Embodiments of the disclosed systems and methods provide hybrid implantable neuromodulator(s) that may deliver combined electrical and pharmacological stimuli to a target anatomical location, e.g., the dorsal root ganglia (DRG), spinal nerve and dorsal root. Such stimuli may function as a non-opioid therapy of chronic pain. The persistence and long-lasting nature of chronic pain may be reversed by blocking sensitized nociceptive afferents, especially C-fiber nociceptors via combined pharmacological and electrical neuromodulation of the DRG, dorsal root and spinal nerve, thereby addressing and reducing chronic pain that may feature maladaptive changes in the central nervous system, which may be driven by sensitized peripheral nociceptors.

For contextual purposes, technical, anatomical and clinical information of relevance to the disclosed systems and methods are briefly described herein.

Generally, the terms “afferent neurons” and “efferent neurons” refer to different types of neurons. Within the peripheral nervous system, afferent neurons perform sensory functions while efferent neurons perform motor functions. Both types of neurons are electrically excitable cells that serve as the structural and functional unit of the nervous system. Generally, a neuron includes a cell body and nerve fibers which extend out from the cell body. Unlike the neurons in the brain and spinal cord, afferent neurons only have axons, but no dendrites. The peripheral axons extend to the end organs to sense the external stimuli, e.g., mechanical brushing, heat, cold, or chemicals. The central axon projects to the spinal cord. Action potentials are generated from the peripheral nerve ending and transmit down the peripheral and central axons to the spinal cord. The cell body of the afferent connects with the peripheral and central axons via a stem axon; and the three axons connect at the T-junction in the DRG. Multiple afferent and efferent axons clustering together in parallel is referred to as “a nerve.”

Afferent neurons carry a sensory signal from sensory receptors of the skin and other organs to the central nervous system (i.e., brain and spinal cord), whereas efferent neurons carry a motor signal away from the central nervous system to the muscles and glands of the body. Afferent and efferent nerve fibers are not connected directly. A third type of neuron, referred to as an “interneuron” or “association” neuron, are neurons within the spinal cord that act as a relay between the afferent neurons and the efferent neurons for communication there between.

Afferent neurons have cell bodies located just outside of the spinal cord in the dorsal root ganglion. Unlike most other neurons, the cell body of an afferent has a single axon that divides into two distinct branches: one connected to the sensory organ and another that carries sensory information to the spinal cord via the dorsal root. Efferent neurons have their cell bodies located in the ventral horn of the spinal cord. From there, efferent axons leave the spinal cord through the ventral root, travel through the spinal nerves, and ultimately synapse with the skeletal muscle cells found in the neuromuscular junction.

Two mechanisms have evolved to transmit nerve signals. First, within cells, electrical signals are conveyed along the cell membrane. Second, for communication between cells, the electrical signals generally are converted into chemical signals conveyed by small messenger molecules called neurotransmitters.

The mechanism underlying signal transmission within neurons is based on voltage differences (i.e., potentials) that exist between the inside and the outside of the cell. This membrane potential is created by the uneven distribution of electrically charged particles, or ions, the most important of which are sodium (Na⁺), potassium (K⁺), chloride (Cl⁻), and calcium (Ca²⁺). Ions enter and exit the cell through specific protein channels in the cell's membrane. The channels “open” or “close” in response to neurotransmitters or to changes in the membrane potential of the cell. The resulting redistribution of electric charge may alter the voltage difference across the membrane. A decrease in the voltage difference is called “depolarization.” If depolarization exceeds a certain threshold, an impulse (i.e., action potential) will travel along the neuron membrane. The inactivation of sodium channels ensure that the action potential propagates in only one direction, toward the axon tip. The generation of an action potential is sometimes referred to as “firing.”

In some embodiments, the disclosed systems and methods deliver peripheral afferent nerve blocking by low-frequency electrical stimulation. Unlike conventional nerve-blocking protocols that implement kilohertz electrical stimulation, the disclosed systems and methods may target unmyelinated C-type and thinly myelinated Aδ-type sensory afferents by low-frequency electrical stimulation (typically <5 Hz at each electrode lead). This low frequency stimulation significantly reduces power consumption to allow long-lasting and on-demand nerve block for suppressing pain in patients.

In some embodiments, the disclosed systems and methods deliver direct sensory transmission block by optimized electrical stimulation. This mechanism of action is different from conventional approaches of evoking paresthesia to block pain in spinal cord stimulation or peripheral nerve stimulation.

In some embodiments, the disclosed systems and methods deliver selective transmission block of unmyelinated C-fibers and slow-conducting Aδ-fibers, the sensitization of which plays critical roles in chronic pain. In an embodiment, the disclosed systems and methods deliver highly selective transmission block of these small-diameter axons through delivery of one or more neuromodulation frequencies of specific afferents that may be determined based on the conduction velocity (CV) of that afferent, which is a quantitative indicator of the afferent diameter.

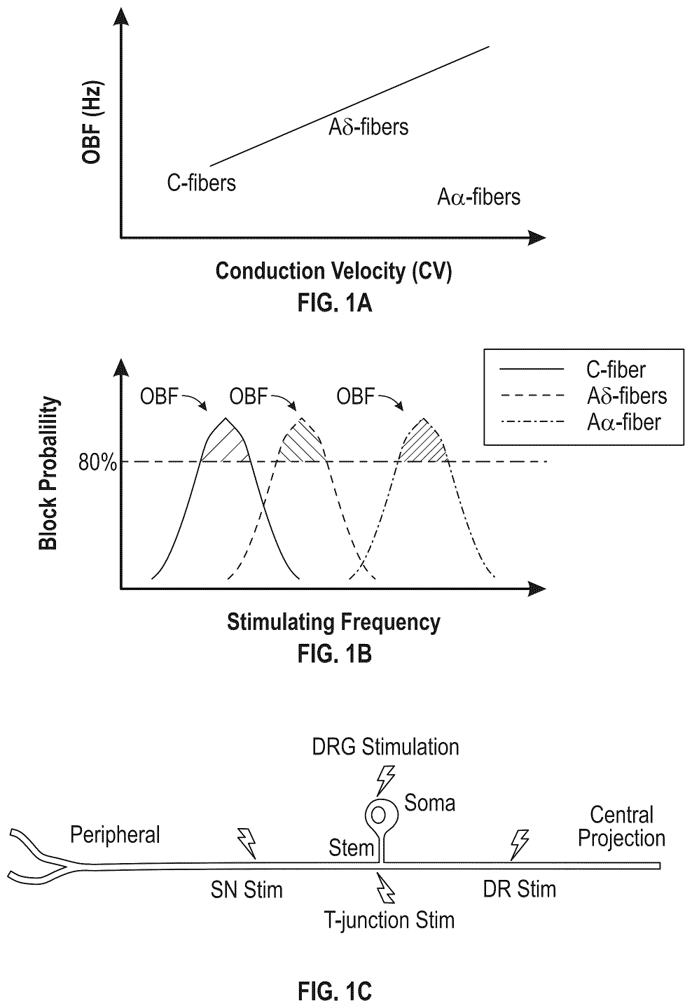

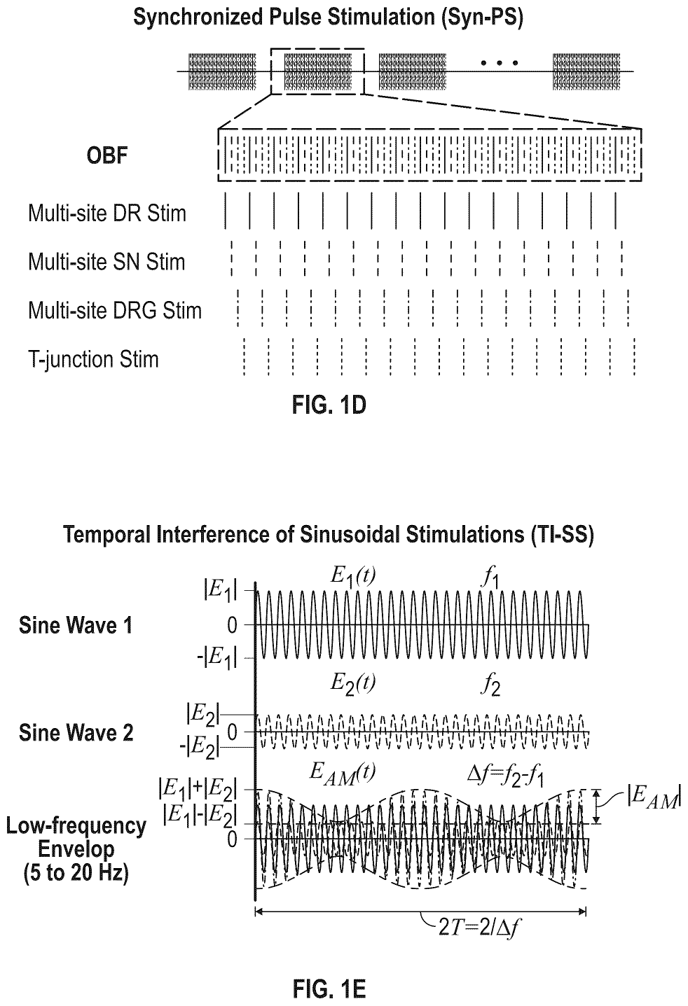

Experimental data has shown an approximately proportional relationship between the optimal blocking frequency (OBF) and CV for afferent nerve signals at the sub-kilohertz frequency range. Frequencies above or below the OBF block a much lower proportion of afferents with that specific CV, or diameter range. As shown in FIG. 1A, optimal blocking frequency (OBF) is approximately proportional to the CV and has the highest rate of conduction block for that particular afferent as shown in FIG. 1B.

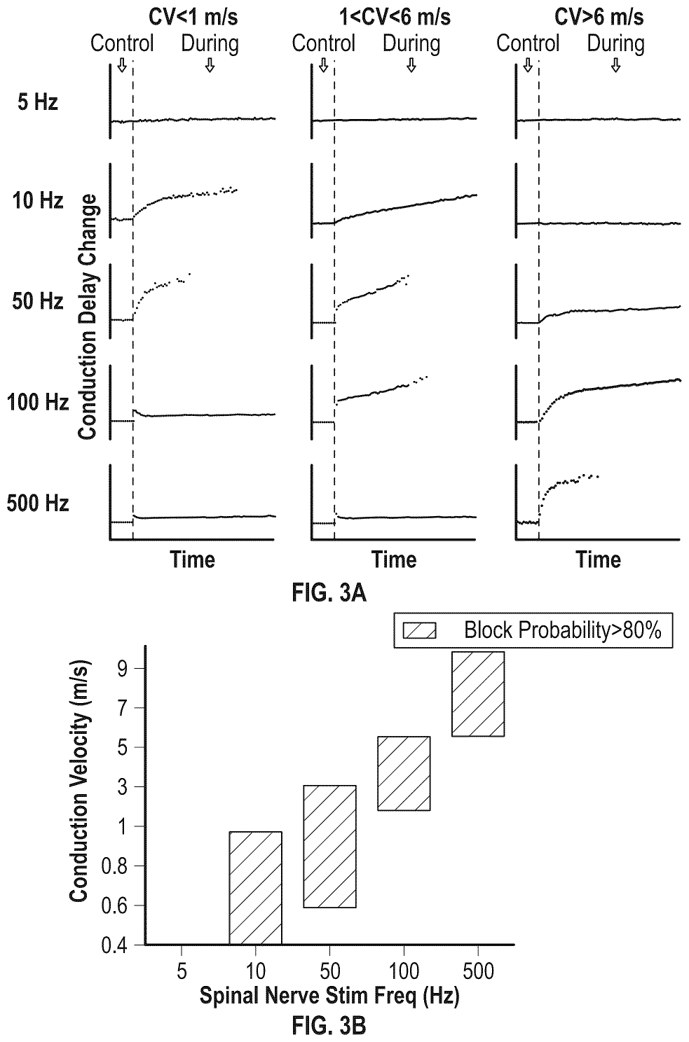

In some embodiments, afferents with different conduction velocity (CV) are blocked by stimulation with different range of frequency. As illustrated in FIG. 1B, an optimal blocking frequency (OBF) may be identified that provides a high likelihood, and potentially highest likelihood, to block afferents with the identified range of CV. A positive correlation exists between the CV of an afferent and the OBF associated with that afferent. For example, as shown in the plots of FIG. 3A, unmyelinated C-fibers with CV less than 1 m/s are optimally blocked by 10- 50 Hz stimulation. Thinly myelinated Aδ-fibers are optimally blocked by 50-100 Hz stimulation. Fibers with CV over 6 m/s are not blocked by electrical stimulations of below 500 Hz. Frequency below 5 Hz does not generally block any afferent transmission. As shown in the plot of FIG. 3B, OBF increases for afferents with greater CV. This indicates that afferents with larger diameter require higher OBF, and low-frequency stimulation (<100 Hz) are selective in blocking small-diameter afferents like the C- and Aδ-fiber nociceptors.

The temporal and spatial summation of multichannel pulsed stimulation allows selective block of a sub-population of C-fibers and slow-conducting Aδ fibers that receive combined neural activities of OBF at their T-junctions from synchronized stimulation at multiple sites on the peripheral nerve, where stimuli at an individual site may be delivered at frequencies much lower than the OBF (typically <5 Hz) as shown in FIG. 1C.

In some embodiments, the disclosed systems and methods deliver temporal interference of sinusoidal stimulations (TI-SS) as shown in FIG. 1E. The two sine waves operate at kilohertz frequencies, which differ slightly, e.g., 2000 and 2020 Hz. The interference caused by these two kilohertz stimulations at the focal region of the DRG, spinal nerve, or dorsal root results in a kilohertz stimulation with a low-frequency envelope equal to the frequency difference between the two source stimuli. The kilohertz signal is filtered out by the neuron due to inward rectification, resulting in a low-frequency sinusoidal stimulation (e.g., 20 Hz for the interference between the 2000 and 2020 Hz stimulations). Afferent fibers with the desired conduction velocity (CV) can be blocked by adjusting the frequency difference to the optimal blocking frequency (OBF) of the fiber. Additionally, the focal region of the temporal interference can be fine-tuned by adjusting the relative amplitudes of the two sinusoidal stimuli. The number of concurrent sinusoidal stimulations is not limited to two. More than two sinusoidal stimulations can be delivered, resulting in multiple temporal interferences to stimulate neuronal tissues at multiple sites simultaneously.

In some embodiments, the disclosed systems and methods deliver temporally controlled and reversible peripheral nerve block(s). As compared to chemical peripheral nerve block by local anesthetics, which offer slow onset and prolonged effects with very little temporal control, embodiments of the disclosed systems and methods achieve sensory transmission block within minutes after the start of electrical stimulation. Also, neural transmission generally resumes within minutes after terminating the stimulation.

Experimental results demonstrate efficacy of the disclosed systems and methods. In embodiments, experimental results demonstrate that low-frequency electrical stimulation of peripheral nerves can block action potential transmission in C- and Aδ-fiber afferent axons.

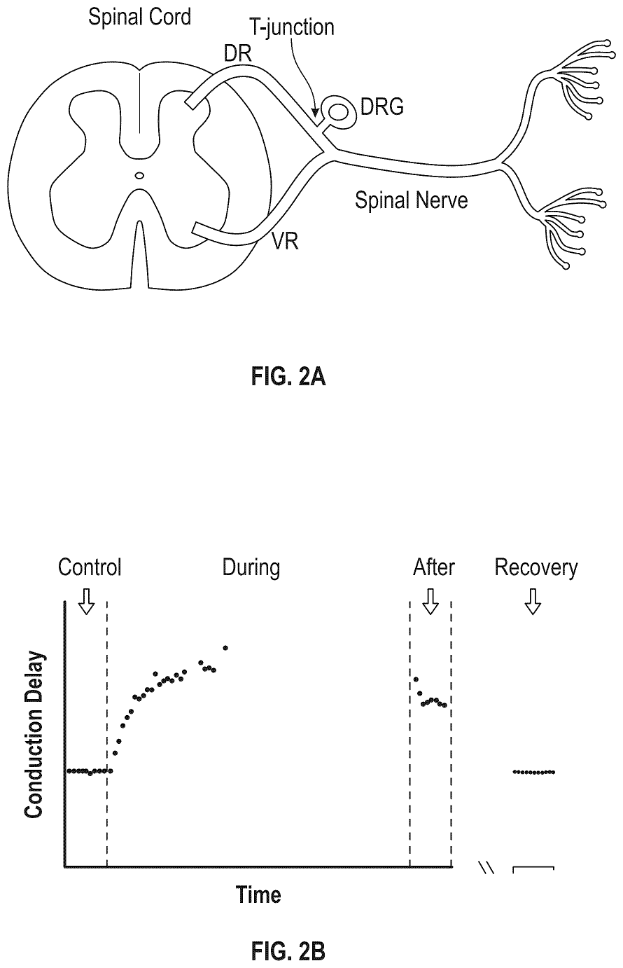

Experimental data reveal that sub-kilohertz peripheral nerve stimulation reversibly blocks neural transmission of sensory afferent fibers. As illustrated in FIG. 2A, sensory afferents are pseudo-unipolar neurons with cell bodies in the dorsal root ganglia (DRG) and two axons projecting peripherally to end organs and centrally to the dorsal spinal cord, respectively. The peripheral axons form a portion of the spinal nerve which also includes axons from motor efferents and autonomic neurons. From recording neural activities at the spinal nerve, the consensus in current literature is that kilohertz electrical stimulation, usually over 10,000 Hz is required to block action potential transmission in peripheral nerves.

Single-fiber recordings to monitor neural activities at the dorsal roots that contain the central axons of the afferents have been performed. The single-fiber recording demonstrated reversible transmission block with sub-kilohertz electrical stimulation as low as 10 Hz delivered at either the DRG or spinal nerve. Displayed in FIG. 2B is a typical example of afferent transmission block by sub-kilohertz electrical stimulation at the spinal nerve. Conduction delay progressively increases at each evoked action potential by the electrical stimulation protocol until complete transmission block. This transmission block appears to take place at the T-junction, i.e., the three-way junction between the peripheral, central and stem axons.

Through computational simulation using a neural membrane model, an accumulation of intracellular sodium concentration at the narrow intra-axonal space, especially near the T-junction region has been identified as a potential underlying mechanism for transmission block. Only small-diameter afferents like the C- and Aδ-fiber nociceptors can be blocked by low-frequency stimulation (<100 Hz) whereas motor efferents with larger diameters cannot.

In some embodiments, spatially and temporally synchronized peripheral nerve stimulation is used to achieve selective afferent block of unmyelinated C-fibers and thinly myelinated Aδ-fibers.

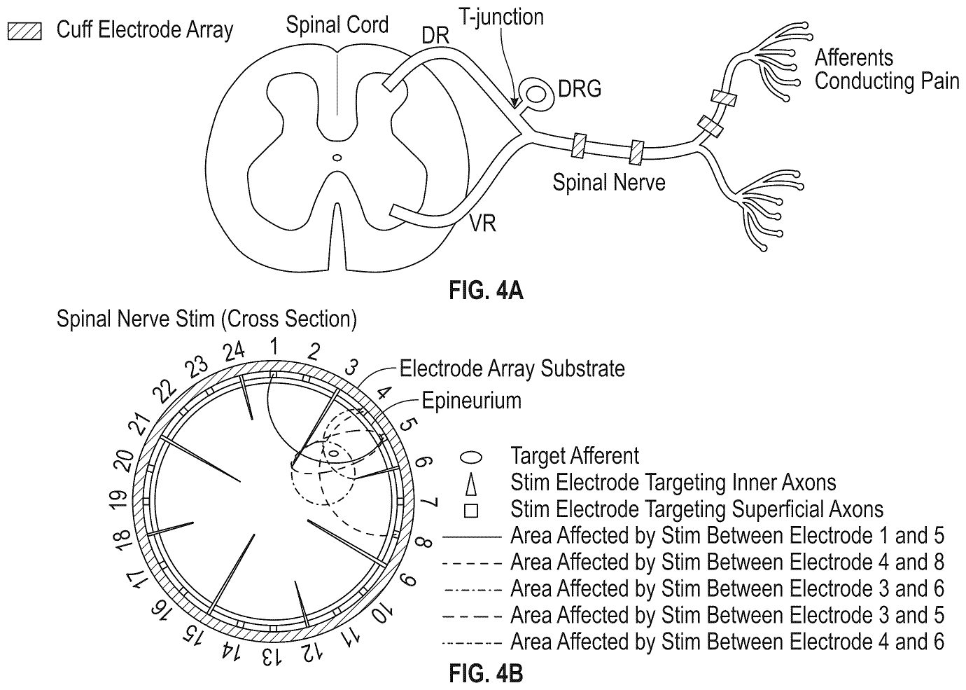

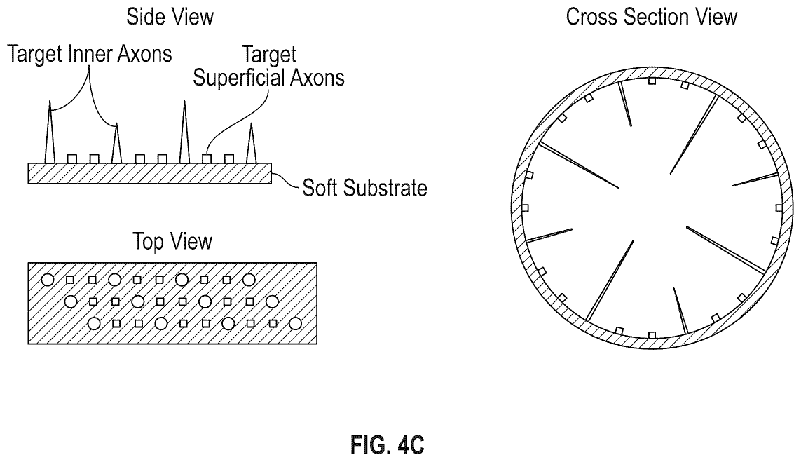

As schematically depicted in FIG. 4A, a nerve cuff electrode may be employed that includes multiple contact leads in the circumference and penetrating needle leads inside the epineurium. Low-frequency electrical stimulation of below 500 Hz may be delivered which will not block motor efferents or any fast-conducting sensory afferents (e.g., Aα- and Aβ-fibers with CV over 6 m/s). In addition, selectivity is enhanced to block only a sub-population of unmyelinated C-fibers and slow-conducting Aδ-fibers. Yet, the blocked afferents do not need to be in close proximity to the stimulating electrode leads.

In some embodiments, three strategies may be used to accomplish spatially selective stimulation.

First, temporally synchronized subthreshold pulse stimuli from multiple stimulating electrodes interfere with each other to form suprathreshold stimuli at the desired location to stimulate the specific afferent, as shown in FIG. 4B. Different combinations of interfered stimuli at a single stimulating site reduce the possibility of off-target side effects via reducing the frequency of stimulation at certain area. FIG. 4C provides schematic depictions of an electrode array layout targeting the peripheral nerve trunk.

Second, increased selectivity is achieved by spatial summation of synchronized pulse stimulation (Syn-PS, typically <5 Hz) at multi-sites along the nerve trunk (see FIG. 4A). From the spatial summation, evoked action potentials will converge at the T-junction of the targeted afferent to achieve spiking frequency at optimal blocking frequency (OBF), which will lead to transmission block of that particular afferent.

Third, suprathreshold sinusoidal stimuli at the OBF can be delivered by temporal interference of sinusoidal stimuli (TI-SS). Two kilohertz sinusoidal stimuli with slight frequency difference causes an amplitude-modulated sinusoidal stimuli at the focal region of interference (see FIG. 1E). The neurons filter out the kilohertz signal through inward rectification of the membrane conductance to receive the low-frequency sinusoidal stimulation, a frequency equal to the frequency difference between the two sinusoidal stimuli.

Superficial electrodes may be fabricated in various geometries, e.g., as rectangular, square, round and/or oval shapes. The size of the electrodes may be adjusted to accommodate different application scenarios. The embedded electrodes may be fabricated with flexible materials, such as carbon fiber, to counteract and/or minimize relative movement between the electrode and nerve tissue.

In some embodiments, a hybrid approach for reversible sensory nerve block implements low-frequency (<20 Hz) electrical stimulation and pharmacological blockage of sodium, potassium ATPase (Na,K-ATPase) at the DRG, which enables selective C-fiber afferent transmission blockage while leaving myelinated A-fibers unaffected. Since sensitized C-fiber afferents drive the chronification of pain, the hybrid approach may serve to reverse the pain chronification by removing the elevated peripheral drive from selectively blocking C-fiber afferents.

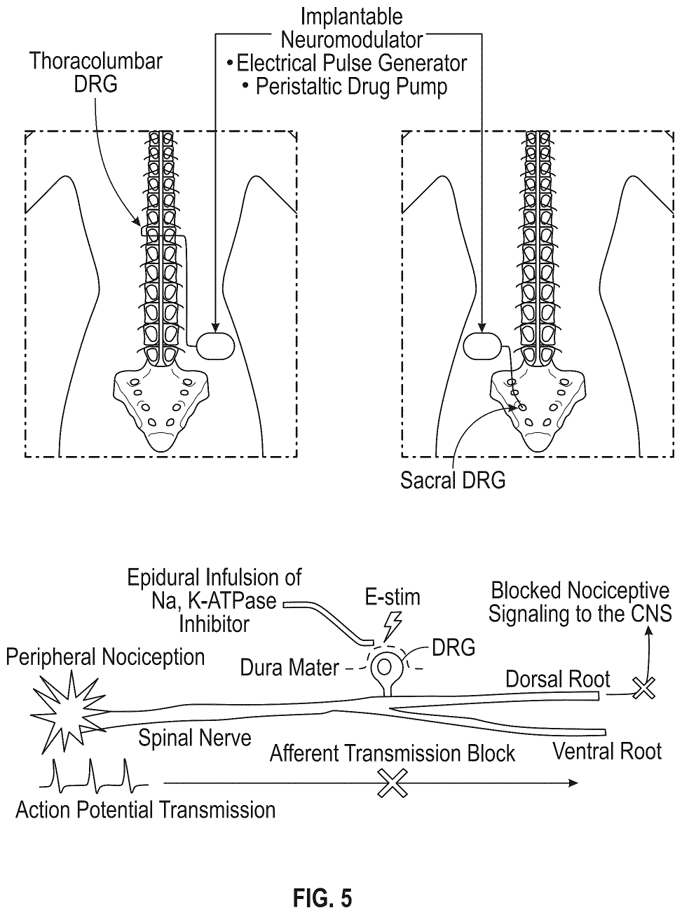

As shown in FIG. 5, an implantable hybrid neuromodulator targets thoracolumbar and sacral DRG to treat chronic pain. The neuromodulator delivers local electrical and pharmacological (inhibitor of the sodium, potassium ATPase, e.g. digoxin) modulation to epidural DRG, achieving reversible afferent transmission block in unmyelinated C-fiber nociceptors. With reference to FIG. 5, “E-stim” refers to electrical stimulation, “CNS” refers to central nervous system, and “Na,K-ATPase” refers to sodium, potassium ATPase.

The hybrid approach delivers selective C-fiber afferent block by sub-kilohertz DRG stimulation while sparing A-fibers, in contrast to the conventional view that kilohertz stimulation is required to reversibly block peripheral neural transmission. In embodiments, the hybrid approach utilizes a Na,K-ATPase inhibitor (e.g., digoxin) that is free of tolerance or addiction associated with conventional pain medications. In embodiments, the hybrid approach directly addresses chronic pain by removing the peripheral drive from sensitized C-fiber nociceptors, potentially normalizing central sensitization, and thereby reversing the chronification of pain. The hybrid electrical/pharmacological neuromodulator addresses unmet clinical need by providing a mechanism-based modality for addressing chronic pain by blocking sensitized C-fiber nociceptors to reverse chronic pain.

In some embodiments, the end users of the hybrid neuromodulator are patients suffering from various types of chronic pain, including chronic leg or arm pain, diabetic neuropathy, failed back surgery syndrome, complex regional pain syndrome, and lower abdominal and pelvic pain. The hybrid neuromodulator can replace existing implantable neurostimulators, including specifically spinal cord stimulators (SCS), and selectively deliver C-fiber transmission block for reversing the disease state of chronic pain.

In embodiments, temporary or permanent reduction of peripheral afferent drives through application of the hybrid neuromodulator can relieve a wide range of chronic pain conditions and can be an efficacious alternative to several invasive medical practices, including peripheral nerve block by local anesthetic injection, peripheral sensory tissue ablation, and thermal block of peripheral nerves.

In some embodiments, a combined pharmacological (Na, K-ATPase antagonists, e.g., digoxin) and electrical neuromodulation of the DRG achieve selective C-fiber afferent transmission blockage while leaving myelinated A-fibers unaffected. This hybrid/combination approach synergistically achieves selective block of sensitized C-fiber nociceptors and may serve to reverse the chronification of pain by removing this elevated peripheral drive.

The hybrid neuromodulator differs significantly from conventional drug or neuromodulatory approaches that only offer temporary suppression of pain, but cannot treat the disease of chronic pain. By reversing the disease state of chronic pain, application of the hybrid neuromodulator increases the quality of life for chronic pain patients, increases the productivity of a large patient population suffering from pain, and mitigates the societal challenge of opioid abuse.

In some embodiments, the combined drug/neuromodulation approach achieves a direct afferent transmission block for chronic pain management. This mechanism of action is different from the conventional neuromodulation approach of evoking paresthesia to block pain, which manages the symptom of pain, but does not treat the disease of chronic pain. In contrast, the hybrid neuromodulator serves to block sensitized C-fiber nociceptors, thereby reversing the disease state of chronic pain.

In some embodiments, the hybrid neuromodulator selectively targets unmyelinated C-fiber afferents, the sensitization of which is the causal factor for many chronic pain symptoms. The hybrid neuromodulator enables selective block of unmyelinated C-fiber afferents while leaving A-fiber afferents and motor efferents carrying normal physiological functions unblocked. This selective block phenomenon significantly reduces off-target side effects to offer focal management of chronic pain through selective neuromodulation of small-diameter nociceptors, especially C-fiber nociceptors while leaving large sensory and motor fibers intact.

In some embodiments, selective targeting of C-fiber nociceptors is achieved by co-applying electrical stimulation with a Na,K-ATPase inhibitor, e.g., digoxin. The afferent transmission block is caused by significant changes in intra-axonal Na⁺ and K⁺ ionic concentrations, which are regulated by Na,K-ATPase.

A lower density of Na,K-ATPase in C-fiber afferents as compared with A-fiber afferents render C-fiber afferents more likely to be blocked by combined Na,K-ATPase inhibition and electrical stimulation.

Blocking the Na,K-ATPase by the antagonist, e.g. digoxin, reduces the stimulation frequency required to block C-fiber nociceptors. The optimal blocking frequency (OBF) for C-fiber afferent is generally between 20 to 50 Hz. The presence of Na,K-ATPase inhibitor “accelerates” the changes in intra-axonal Na⁺ and K⁺ concentrations and enables afferent block at a much lower stimulus frequency (e.g., <10 Hz).

The presence of a Na,K-ATPase antagonist prolongs the recovery of afferent transmission after terminating the electrical stimulation, allowing intermittent electrical stimulation to maintain the afferent transmission block. Compared with existing neuromodulators that generally operate continuously at much higher frequencies (some even at 10 kHz), the hybrid neuromodulator operating intermittently at low stimulus frequency (e.g., <10 Hz) consumes significantly less power and allows a more flexible design of the power system.

Applying Na,K-ATPase antagonist, e.g. digoxin locally in the dorsal root ganglia (DRG) has minimal systemic side effects. Digoxin is a plant-derived steroid-like compound widely used for the treatment of congestive heart failure in the clinic. Serum digoxin levels of <1.0 ng/ml in patients are considered safe with no apparent side effects. In embodiments, the bimodal neuromodulation technique allows prolonged transmission block. The presence of a Na,K-ATPase inhibitor in the intervertebral foraminal canal increases the duration to resume transmission by retarding the recovery process. Furthermore, the release of Na,K-ATPase can be prolonged to more than fourteen (14) days by encapsulating into drug delivery vehicles, e.g., liposomes and poly(lactic-co-glycolic acid) (PLGA) microspheres.

Efficacy of the hybrid neuromodulator treatment regimen is supported by various experimental results. First, it has been demonstrated that sub-kilohertz DRG stimulation (e.g., 10-100 Hz for C-fibers) reversibly blocks afferent transmission via activity-dependent conduction slowing.

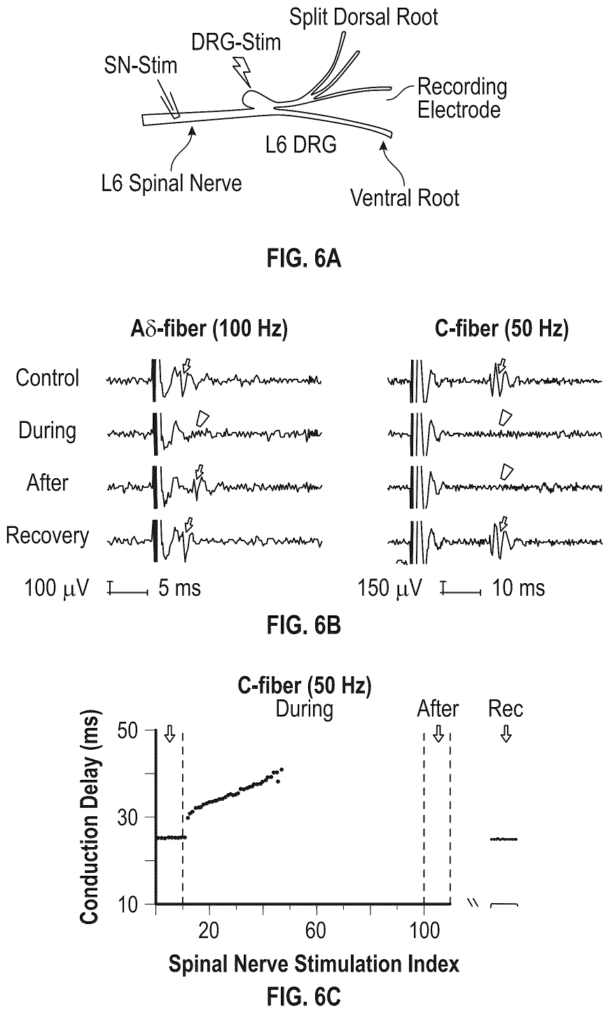

As shown in FIGS. 6A-6C, sub-kilohertz DRG stimulation is effective in reversibly blocking afferent transmission via activity-dependent conduction slowing In an ex vivo preparation with spinal nerve, DRG, dorsal and ventral roots in continuity (see FIG. 6A), action potentials (AP) evoked from the peripheral spinal nerve failed to transmit to the central dorsal root when sub-kilohertz electrical pulse stimulation was delivered to the DRG (see FIG. 6B). The conduction delay measured once every 2 sec showed a progressive increase trend following the onset of DRG stimulation, which eventually leads to conduction block (see FIG. 6C). Afferent transmission remained blocked for 5-15 min after terminating the DRG stimulation and eventually recovered to pre-stimulus conduction velocity (see FIG. 6C). The optimal blocking frequency (OBF) for C-fibers is shown to be ≤50 Hz, lower than the 100 Hz OBF for thinly myelinated Aδ-fibers. OBF for fast-conducting Aβ- and Aα-fibers appears to be beyond 500 Hz. This differential makes it possible to selectively block slow-conducting C-and Aδ-fibers while sparing fast-conducting Aβ- and Aα-fibers when stimulating the DRG at a frequency below 100 Hz.

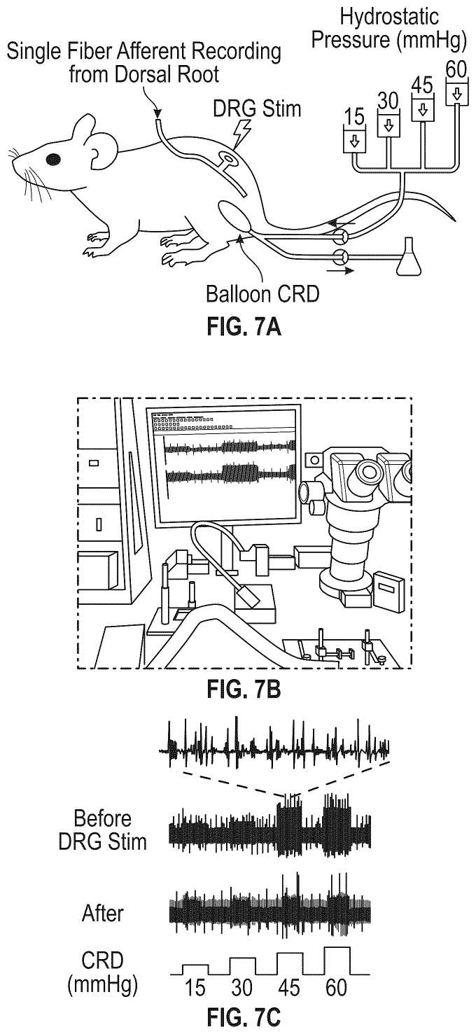

With reference to FIGS. 7A-7C, in vivo single-fiber recordings from the L6 dorsal root in an isoflurane-anesthetized mice were obtained. Action potentials were evoked by graded balloon colorectal distension (CRD; 15-60 mmHg). The L6 DRG was exposed to allow electrical DRG stimulation (50 Hz, 120% of the motor threshold). Using in vivo recordings from the L6 dorsal root of a mouse (as schematically and photographically depicted in FIGS. 7A and 7B, respectively), it was demonstrated that sub-kilohertz DRG stimulation blocked visceral afferent transmission (see FIG. 7C).

The change in intracellular Na⁺ and K⁺ concentrations by a single action potential spike is trivial in the neural somata (<1 mM) due to the large volume-to-surface ratio. However, in an unmyelinated C-fiber axon with diameter of ˜1 μm, the volume-to-surface ratio is reduced by ˜1 order of magnitude compared with the soma, resulting in significant changes in intracellular Na⁺ and K⁺ concentrations following each AP, i.e., by 2 to 4 mM according to a computational modeling.

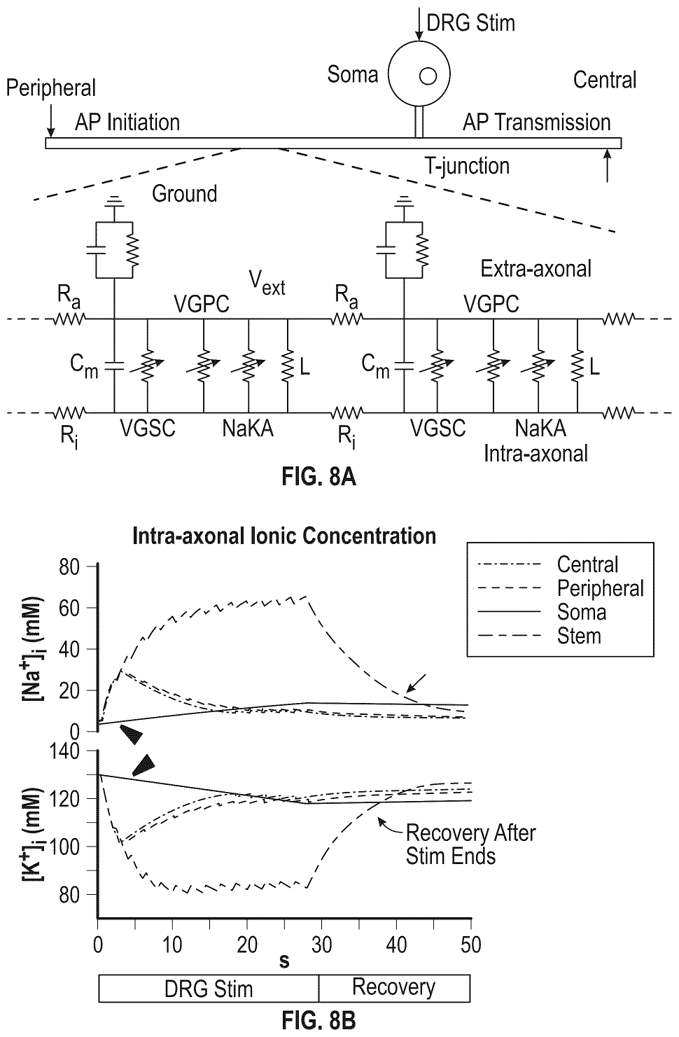

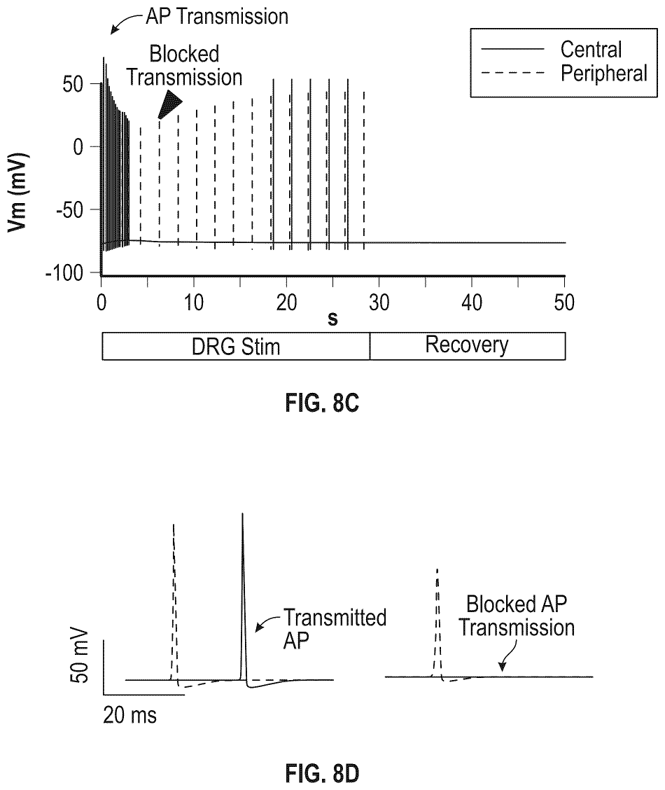

FIGS. 8A-8D relate to computational modeling simulation of DRG stimulation in an unmyelinated C-fiber afferent. FIG. 8A shows a neural membrane model that includes a soma, stem axon, T-junction, and peripheral and central axons. DRG stimulation is delivered extracellularly 5 μm away from the soma. Intracellular Na⁺ and K⁺ concentrations are modeled to be affected by Na,K-ATPase, transmembrane ionic current flow and axial ionic diffusion. The computational modeling shows that 20 Hz DRG stimulation can lead to progressive increase in intra-axonal Na⁺ concentration and reduction of intra-axonal K⁺ concentration at afferent axons close to the T-junction between the stem, central and peripheral axons (see FIG. 8B).

Na⁺ and K⁺ concentrations cannot be rapidly restored by Na,K-ATPase. Simulation shows that applying 20 Hz DRG stimulation to a C-fiber afferent can cause a significant reduction of transmembrane Na⁺ and K⁺ gradients. This directly leads to an AP transmission block at axonal regions close to the T-junction (see FIG. 8C).

The computational simulation results in FIGS. 8A-8D show progressive disruption of the transmembrane Na⁺ and K⁺ ionic gradient in axonal segments around the T-junction following rapid AP spiking from DRG stimulation. This disrupted ionic gradient can be restored by elevated Na,K-ATPase activity after terminating DRG stimulation. Because Na,K-ATPase is a membrane-bound enzyme that works against the transmembrane Na⁺ and K⁺ concentration by exporting Na⁺ extracellularly and importing K⁺ intracellularly, pharmacological inhibition of Na,K-ATPase can serve to prolong the afferent transmission block by decreasing the rate of restoring the Na⁺ and K⁺ ionic gradient.

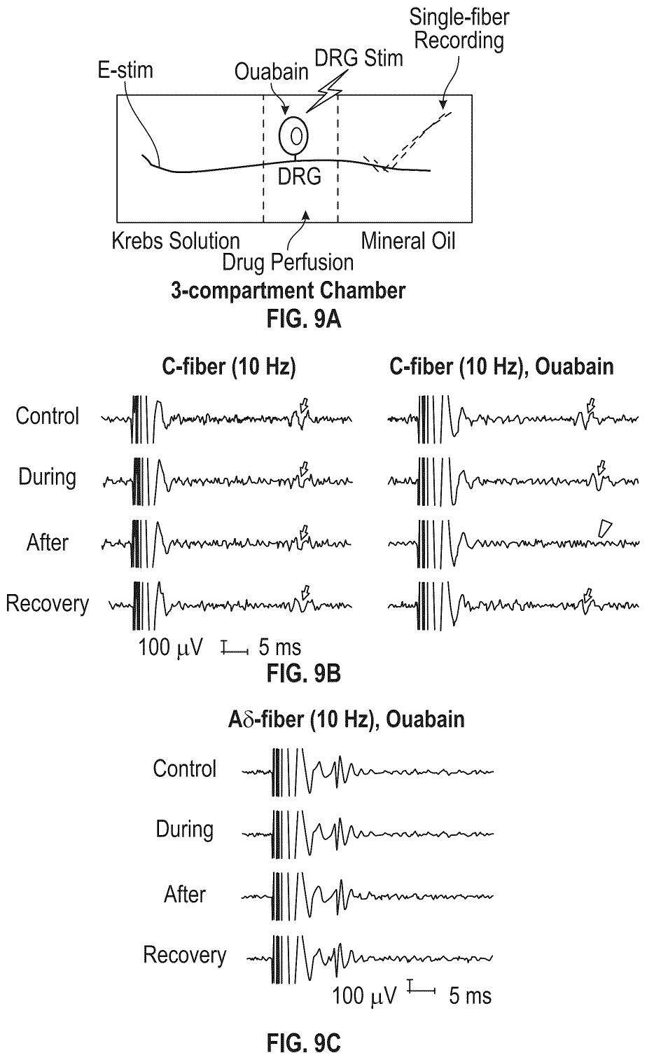

As shown in FIGS. 9A-9D, the effect of Na,K-ATPase inhibition (using ouabain as an antagonist) on afferent transmission block by DRG stimulation was assessed and demonstrated that Na,K-ATPase inhibition by ouabain significantly enhanced the efficacy of afferent transmission block by DRG stimulation. As shown in FIG. 9A, a 3-compartment ex vivo tissue chamber was used to separate the DRG from the spinal nerve and dorsal root. Action potentials were evoked by stimulating the peripheral end of the spinal nerve, which is transmitted down the peripheral and central axons to be recorded by extracellular single-fiber recordings from split dorsal root filaments. Local application of ouabain (10 μg/mL or 17.1 μM) to the DRG in the middle compartment significantly prolonged transmission block by DRG stimulation as measured by the time to resume transmission following terminating the DRG stimulation. The ouabain concentration is below the Kd to block the α1 unit of Na,K-ATPase (32-170 μM). DRG stimulation at 10 Hz, much lower than the optimal blocking frequency (OBF) for C-fibers (˜50 Hz), effectively blocks afferent transmission in the presence of ouabain (10 μg/mL) (see FIG. 9B). Ouabain also led to a greater separation of OBF between C-fibers and thinly myelinated Aδ-fibers. The test results demonstrate that 10 Hz electrical stimulation selectively blocks a C-fiber afferent (FIG. 9B) while leaving an Aδ-fiber unblocked (FIG. 9C).

Ouabain belongs to a large family of plant-derived steroid-like compounds (cardiac glycosides) which are potent Na,K-ATPase inhibitors and widely used for the treatment of congestive heart failure. By blocking Na,K-ATPase, cardiac glycosides increase intracellular Na⁺ concentration in cardiac myocytes, which increases intracellular Ca²⁺ concentration by decreasing the activity of a Na⁺/Ca²⁺ exchanger. Increased intracellular calcium leads to an increased magnitude of myocardial contractility, which addresses irregular heartbeat and heart failure. Digoxin and digitoxin have been extensively reported in pharmacological studies in human subjects and are commercially available for human and animal use. Digitoxin and digoxin have comparable mechanisms of action, differing in their routes of elimination from the body. Digoxin is eliminated via the kidneys and digitoxin is eliminated via the liver. Serum digoxin levels at <1.0 ng/ml are considered safe with no apparent side effect. In embodiments, digoxin is used in the hybrid neuromodulator.

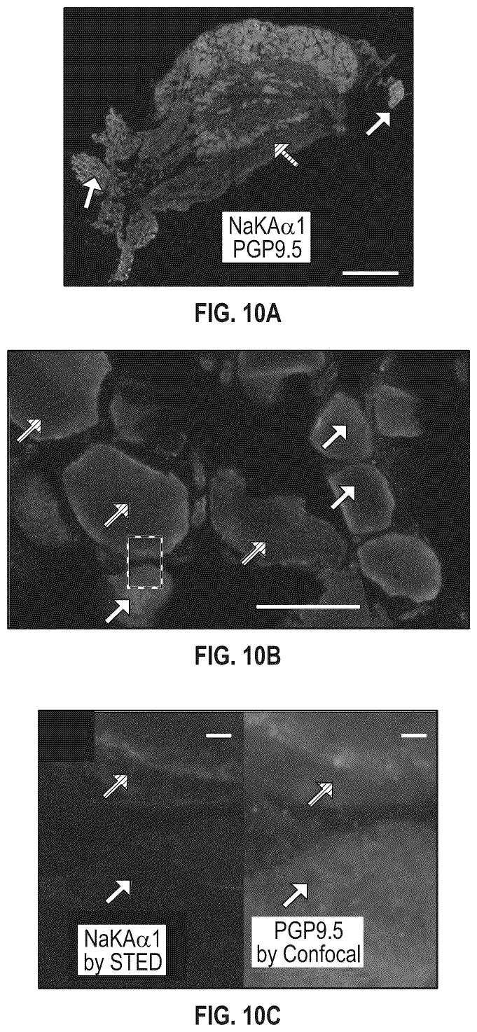

FIGS. 10A-10C show expression of Na,K-ATPase in the DRG and that, based on antibody against the α1 subunit of Na,K-ATPase (NaKAα1) stained DRG sections, revealed significantly higher expression of NaKAα1 in axons outside the DRG (non-cross-hatched arrows) than axons within the DRG (cross-hatched arrow). Magnified view (60×) reveals that NaKAα1 expression linearly correlates with the DRG neural size, i.e., larger neurons show greater immunostaining intensity of NaKAα1 than smaller neurons (see FIGS. 10B and 10C).

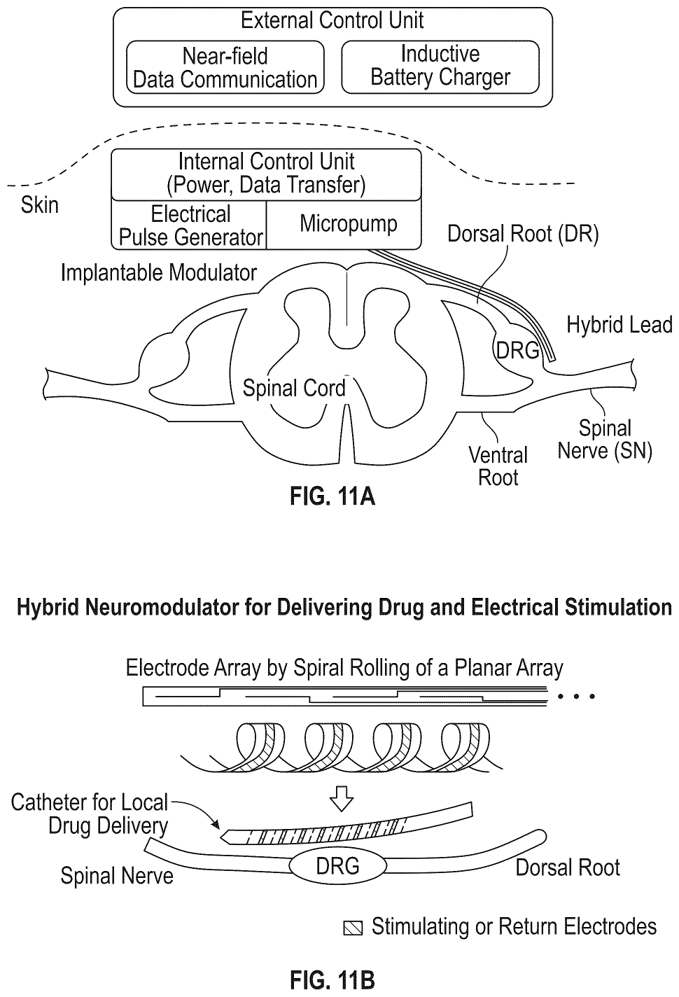

Schematics of a hybrid electrical/pharmacological neuromodulator that targets the DRG to treat chronic pain are provided in FIGS. 11A and 11B.

With reference to FIG. 11A, a schematic depiction of a system for delivery of DRG stimulation is provided. In embodiments the hybrid neuromodulator includes a hybrid lead that delivers multichannel electrical stimulation and epidural Na,K-ATPase antagonist, e.g. digoxin infusion, to the DRG. In embodiments, the hybrid neuromodulator may include a subcutaneous implantable modulator for electrical pulse generation and fluid infusion via a peristaltic micro-pump. In embodiments, the hybrid neuromodulator includes an external control unit outside the body, which communicates with the implantable modulator via near-field wireless communication for data and command transmission. In embodiments, the hybrid neuromodulator may include an external inductive battery charger. The hybrid neuromodulator may also include and/or cooperate with additional ancillary components. A schematic of a hybrid electrical/pharmacological neuromodulator that targets the DRG to treat chronic pain is provided in FIG. 11A.

The external control unit is adapted to communicate with an internal control unit that receives power and data transfer. The data transfer includes both receipt of data, e.g., from the external control unit, and transmission of data, e.g., to the external control unit. The internal control unit includes an electrical pulse generator and a micropump for delivery of stimulation energy to a desired anatomical location. The internal control unit is in electrical communication with a lead, e.g., a hybrid lead, that delivers stimulation energy to a patient. The internal control unit may take the form of an implantable modulator, i.e., for implantation below the skin.

Turning to the design of the hybrid neuromodulator as schematically depicted in FIG. 11B, in embodiments the hybrid neuromodulator includes a hybrid lead that delivers multichannel electrical stimulation and epidural Na,K-ATPase antagonist, e.g. digoxin infusion, to the DRG. In some embodiments, the hybrid leads for delivering both electrical and pharmacological neuromodulation can be fabricated by spiral wrapping a micro-electrode array around a fluid-delivery catheter (see FIG. 11B). The micro-electrode array may be fabricated by planar fabrication to have multiple rectangular-shaped electrode leads. By spiral wrapping the planar electrode array onto a catheter, each electrode may be spaced by 30 to 200 microns, allowing adjustable electrode density in contact with the DRG, dorsal root and spinal nerve.

In some embodiments, the hybrid neuromodulator may include a subcutaneous implantable modulator for electrical pulse generation and fluid infusion via a peristaltic micro-pump. In embodiments, the hybrid neuromodulator may include a wireless remote controller. In embodiments, the hybrid neuromodulator may include an external inductive battery charger. The hybrid neuromodulator may also include and/or cooperate with additional ancillary components.

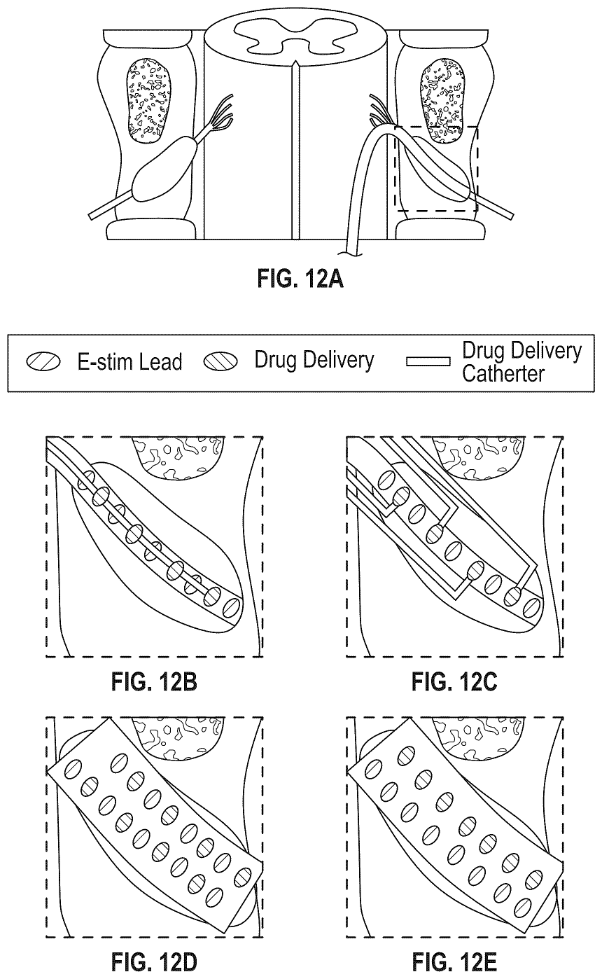

In some embodiments, implantation of the hybrid lead may be undertaken according to conventional techniques, e.g., a procedure that corresponds to the implantation technique for FDA-approved DRG and spinal cord stimulators, e.g., via fluoroscopy-guided insertion through a small opening at the lamina of the dorsal vertebrae (see FIG. 12A).

In some embodiments, the hybrid lead for combined delivery of electrical and pharmacological neuromodulation may be configured to be aligned in a single line along the axis of the DRG (see FIGS. 12B and 12C) or in two lines (see FIGS. 12D and 12E). The electrical stimulation leads can be configured to allow bipolar stimulation between any two pairs of electrodes. The drug delivery leads may be connected to a reservoir containing Na,K-ATPase antagonist (e.g., digoxin) through a single catheter or multiple individual catheters, as shown in FIGS. 12B and 12C.

In some embodiments, the wires and drug catheter may be tunneled to the implantable modulator that is positioned under the skin in the buttock region. The implantable modulator generally consists of two functional units for generating electrical pulses and digoxin delivery, respectively (see FIGS. 11A and 11B). The electrical pulse generation may operate in a conventional manner, e.g., in the manner that off-the-shelf implantable pulse generators (IPG) used in commercial neurostimulators operate.

In some embodiments, microliter drug delivery is provided by a micro-pump. The micro-pump can operate through motor-driven peristalsis, osmotic pressure, thermal expansion via fluid-gas phase change, and various other mechanisms. The confined epidural space inside the vertebral foramen will not dilute the Na,K-ATPase antagonist (e.g. digoxin) excessively and, in embodiments, a daily delivery of microliters of pharmaceuticals may be used to achieve a desired therapeutic concentration at the DRG (e.g., 10 μM of digoxin).

To prolong the effect of the pharmacological block, the pharmacological block (e.g., digoxin) may be packaged into liposome(s) or poly(lactic-co-glycolic acid) (PLGA) microspheres. A micro-pump with a 2 mL reservoir may operate for six (6) months or longer, a period of time that may be sufficient to reverse pain chronification. Refill is generally not necessary, but can be achieved by transcutaneous needle injection.

A remote controller and an inductive battery charge may be positioned outside the skin and may be utilized in similar manner to conventional implantable neurostimulators.

In some embodiments, a system is provided that includes a computer-readable storage medium that stores instructions that, when executed by a computer, cause the computer to effect a reversible sensory block. The reversible sensory block may be effected by a hybrid neuromodulator that includes a hybrid lead for delivering electrical stimulation and pharmacological stimulation.

In some embodiments, a system is provided for effecting a reversible sensory block that includes (i) a hybrid neuromodulator that includes a hybrid lead for delivering electrical stimulation and pharmacological stimulation, and (ii) a controller for controlling delivery of electrical stimulation and pharmacological stimulation by the hybrid neuromodulator. The controller may control delivery of the electrical stimulation based on conduction velocity of an afferent. The controller may control delivery of different frequencies of the electrical stimulation to different afferents with different conduction velocities.

In some embodiments, a system is provided in which the controller may control temporally synchronized stimuli from multiple stimulating electrodes that interfere with each other to stimulate a specific afferent. The system controller may control delivery of a spatial summation of synchronized low-frequency stimulation at multiple sites along a nerve trunk. In embodiments, the spatial summation may translate to evoked action potentials that converge at a T-junction of the targeted afferent.

In some embodiments, a system is provided in which a controller controls delivery of the pharmacological block to the Na,K-ATPase at the dorsal root ganglia. The pharmacological block may be a Na,K-ATPase antagonist or inhibitor. The pharmacological block may be packaged into one or more liposomes or poly(lactic-co-glycolic acid) (PLGA) microspheres.

In some embodiments, a system is provided to deliver a reversible sensory block for treatment of chronic pain. The chronic pain may consist of chronic leg or arm pain, diabetic neuropathy, failed back surgery syndrome, complex regional pain syndrome, lower abdominal pain, and/or pelvic pain.

All statements herein reciting principles, aspects, and embodiments of the disclosure, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It should be noted that terms such as “optimal” which may be construed as a superlative, are not limiting of the teachings herein. That is, for example, the “optimal blocking frequency” (OBF) may apply for a given embodiment, instance, subject and/or set of conditions, but not for others. It should be recognized that OBF and other similar terminology is subject to variability, including, for example, biological variability, and therefore such terminology is used only for purposes of discussion and is not to be construed as limiting of the teachings herein.

Various other components may be included and called upon for providing for aspects of the teachings herein. For example, additional materials, combinations of materials and/or omission of materials may be used to provide for added embodiments that are within the scope of the teachings herein. Adequacy of any particular element for practice of the teachings herein is to be judged from the perspective of a designer, manufacturer, seller, user, system operator or other similarly interested party, and such limitations are to be perceived according to the standards of the interested party.

In the disclosure hereof any element expressed as a means for performing a specified function is intended to encompass any way of performing that function including, for example, a) a combination of circuit elements and associated hardware which perform that function or b) software in any form, including, therefore, firmware, microcode or the like as set forth herein, combined with appropriate circuitry for executing that software to perform the function. Applicant thus regards any means which can provide those functionalities as equivalent to those shown herein. No functional language used in claims appended herein is to be construed as invoking 35 U.S.C. § 112(f) interpretations as “means-plus-function” language unless specifically expressed as such by use of the words “means for” or “steps for” within the respective claim.

When introducing elements of the present invention or the embodiment(s) thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements. The term “exemplary” is not intended to be construed as a superlative example but merely one of many possible examples.

1. A system for effecting a reversible sensory block, comprising:
a hybrid neuromodulator that includes a hybrid lead for delivering electrical stimulation and pharmacological stimulation.

2. The system according to claim 1, further comprising an implantable modulator.

3. The system according to claim 2, wherein the implantable modulator is adapted for electrical pulse generation and pharmacological fluid infusion.

4. The system according to claim 3, further comprising a pump that delivers the fluid infusion.

5. The system according to claim 4, wherein the pump is a micro pump configured to harness a mechanism selected from the group consisting of motor-drive peristalsis, osmotic pressure, and thermal expansion via fluid-gas phase change.

6. The system according to claim 1, wherein the hybrid lead is associated with a nerve cuff.

7. The system according to claim 1, further comprising a wireless remote controller adapted to communicate with the hybrid neuromodulator.

8. A method for effecting a reversible sensory block, comprising:
delivering low-frequency electrical stimulation to unmyelinated C-type and thinly myelinated Aδ-type sensory afferents.

9. A computer-readable storage medium storing instructions that when executed by a computer cause the computer to perform the method of claim 8.

10. A system for effecting a reversible sensory block, comprising:
a hybrid neuromodulator that includes a hybrid lead for delivering electrical stimulation and pharmacological stimulation; and
a controller for controlling delivery of electrical stimulation and pharmacological stimulation by the hybrid neuromodulator.

11. The system according to claim 10, wherein the controller controls delivery of the electrical stimulation based on conduction velocity of an afferent.

12. The system according to claim 11, wherein the controller controls delivery of different frequencies of the electrical stimulation to different afferents with different conduction velocities.

13. The system according to claim 10, wherein the controller controls temporally synchronized stimuli from multiple stimulating electrodes that interfere with each other to stimulate a specific afferent.

14. The system according to claim 10, wherein the controller controls delivery of a spatial summation of synchronized low-frequency stimulation at multiple sites along a nerve trunk.

15. The system according to claim 14, wherein the spatial summation translates to evoked action potentials that converge at a T-junction of the targeted afferent.

16. The system according to claim 10, wherein the controller controls delivery of the pharmacological block to the sodium, potassium ATPase at the dorsal root ganglia.

17. The system according to claim 10, wherein the pharmacological block is a Na, K-ATPase antagonist or inhibitor.

18. The system according to claim 10, wherein the pharmacological block is packaged into one or more liposomes or poly(lactic-co-glycolic acid) (PLGA) microspheres.

19. The system according to claim 10, wherein the reversible sensory block is used for treatment of chronic pain selected from the group consisting of chronic leg or arm pain, diabetic neuropathy, failed back surgery syndrome, complex regional pain syndrome, lower abdominal pain, and pelvic pain.

20. The system according to claim 10, wherein the reversible sensory nerve block is used to treat at least one of neurogenic chronic cough, obesity, fecal incontinence, and urinary incontinence.
